(19) United States
(12) Patent Application Publication
(10) Pub. No.: US 2025/0046414 A1
(43) Pub. Date: Feb. 6, 2025

(54) METHOD AND DEVICE FOR GENERATING CUSTOMIZED DIGITAL THERAPEUTIC BASED ON MULTI-SOURCE DATA ANALYSIS

(71) Applicant: WELT CORP., LTD, Seoul (KR)

(72) Inventors: Seong Ji KANG, Seoul (KR); Hye Kang ROH, Seoul (KR); Joo Young KIM, Seoul (KR); Do Hyun LEE, Gyeonggi-do (KR); Hwa Young JEONG, Incheon (KR)

(21) Appl. No.: 18/457,513

(22) Filed: Aug. 29, 2023

(30) Foreign Application Priority Data
Aug. 4, 2023 (KR) 10-2023-0102206

(51) Int. Cl. G16H 20/00; G16H 10/60; G16H 50/20

(52) U.S. Cl. CPC G16H 20/00; G16H 10/60; G16H 50/20

(57) ABSTRACT

A method of generating a customized digital therapeutic on the basis of multi-source data analysis and an apparatus for performing the same can include receiving, by a digital therapeutic generation device, user data and generating, by the digital therapeutic generation device, a digital therapeutic personalized for a user on the basis of the user data.

### BACKGROUND

### 1. Field of the Invention

The present invention relates to a method of generating a customized digital therapeutic on the basis of multi-source data analysis and an apparatus for performing the same. More specifically, the present invention relates to a multi-source data analysis-based customized digital therapeutic generation method for generating a digital therapeutic by combining various modules for treatment and an apparatus for performing the same.

### 2. Discussion of Related Art

With the development of various smart technologies, data of personal daily activities is recorded, and individual life can be efficiently managed on the basis of the recorded data. In the meantime, health-related data logging is attracting attention due to the increasing interest in healthcare. Many users have already been generating and utilizing various health-related data including data on exercise, diet, sleep, and the like through user devices such as smartphones, wearable devices, and the like. In the past, health-related data was generated and managed only by medical institutions, but now users have begun to generate and manage their own health-related data through user devices such as smartphones and wearable devices.

In many cases, health-related data logging is performed through a wearable device. A wearable device is a user device that is carried by or attached to a user. Due to the development of Internet of things (IoT) and the like, wearable devices are frequently used for collecting health-related data. A wearable device may collect a user's physical change information and surrounding data of the user through equipment and provide advice required for the user's healthcare on the basis of the collected data.

A user's health-related data may include a user biomarker, and research is ongoing on a method of making a medical prescription adaptively to a user on the basis of the user's health-related data.

The present application was developed through the bio-medical technology commercialization support project (BT210029) “Development of Precise Digital Therapeutics for Alcoholism” by Seoul Business Agency in 2021.

As related art, there is Korean Patent No. 10-2425479.

### SUMMARY OF THE INVENTION

The present invention is directed to solving all the foregoing problems.

The present invention is also directed to developing a digital therapeutic by combining a plurality of digital therapeutic modules which may provide a digital treatment service.

The present invention is also directed to controlling collision between treatment data of a plurality of types of digital therapeutics so that a user using a plurality of types of digital therapeutics may receive a treatment service in order of priority without collision between treatment data.

A representative configuration of the present invention for achieving the above objects is as follows.

According to an aspect of the present invention, there is provided a method of generating a customized digital therapeutic on the basis of multi-source data analysis comprises receiving, by a digital therapeutic generation device, user data and generating, by the digital therapeutic generation device, a digital therapeutic personalized for a user on the basis of the user data.

Meanwhile, the digital therapeutic generation device is generated on the basis of a combination of one or more digital therapeutic modules, the digital therapeutic modules include a digital therapeutic module (diagnosis/monitoring) and a digital therapeutic module (treatment).

Further, the digital therapeutic generation device is generated in consideration of a probability of a collision between digital therapeutics or characteristics of user data providable by the user.

According to another aspect of the present invention, there is provided a digital therapeutic generation device for generating a customized digital therapeutic on the basis of multi-source data analysis performs the operations of: receiving user data and generating a digital therapeutic personalized for a user on the basis of the user data.

Meanwhile, the digital therapeutic generation device is generated on the basis of a combination of one or more digital therapeutic modules, the digital therapeutic modules include a digital therapeutic module (diagnosis/monitoring) and a digital therapeutic module (treatment).

Further, the digital therapeutic generation device is generated in consideration of a probability of a collision between digital therapeutics or characteristics of user data providable by the user.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a conceptual diagram of a digital therapeutic generation system according to an exemplary embodiment of the present invention.

FIG. 2 is a conceptual diagram illustrating operations of a digital therapeutic generation device according to an exemplary embodiment of the present invention.

FIG. 3 is a conceptual diagram illustrating a method of generating a digital therapeutic according to an exemplary embodiment of the present invention.

FIG. 4 is a conceptual diagram illustrating a method of generating a personalized digital therapeutic according to an exemplary embodiment of the present invention.

FIG. 5 is a conceptual diagram illustrating a digital therapeutic generation method according to an exemplary embodiment of the present invention.

FIG. 6 is a conceptual diagram illustrating a method of managing a digital therapeutic for a user according to an exemplary embodiment of the present invention.

### DETAILED DESCRIPTION OF EXEMPLARY EMBODIMENTS

The detailed description of the present invention will be made with reference to the accompanying drawings showing examples of specific embodiments of the present invention. These embodiments will be described in detail such that the present invention can be performed by those skilled in the art. It should be understood that various embodiments of the present invention are different but are not necessarily mutually exclusive. For example, a specific shape, structure, and characteristic of an embodiment described herein may be implemented in another embodiment without departing from the scope and spirit of the present invention. In addition, it should be understood that a position or arrangement of each component in each disclosed embodiment may be changed without departing from the scope and spirit of the present invention. Accordingly, there is no intent to limit the present invention to the detailed description to be described below. The scope of the present invention is defined by the appended claims and encompasses all equivalents that fall within the scope of the appended claims. Like reference numerals refer to the same or like elements throughout the description of the figures.

Hereinafter, in order to enable those skilled in the art to practice the present invention, exemplary embodiments of the present invention will be described in detail with reference to the accompanying drawings.

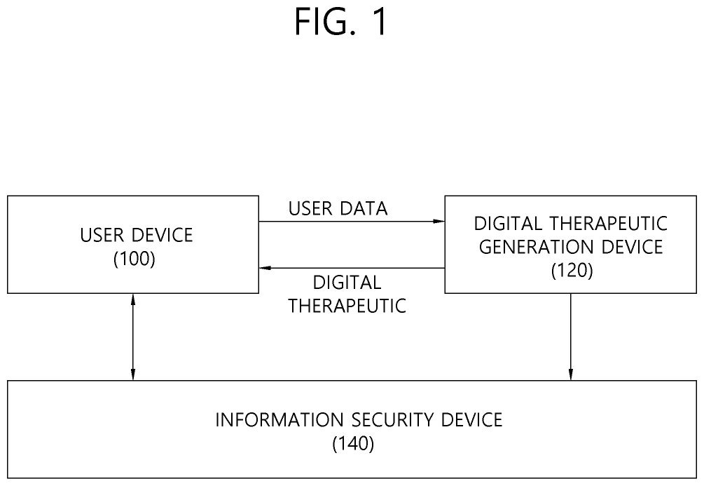

FIG. 1 is a conceptual diagram of a digital therapeutic generation system according to an exemplary embodiment of the present invention.

FIG. 1 shows a digital therapeutic generation system for generating a digital therapeutic.

Referring to FIG. 1, the digital therapeutic generation system may include a user device 100, a digital therapeutic generation device 120, and an information security device 140.

The user device 100 may generate user data for generating a digital therapeutic for a user, transmit the user data to the digital therapeutic generation device 120, and receive a digital therapeutic which is determined adaptively to the user.

The user data may include various data used for generating the digital therapeutic for the user. For example, the user data may include biomarker data acquired through the user device 100 (e.g., a smartphone or a wearable device), input data separately input by the user, and data of the user's personal information (sex, age, and the like).

For example, a digital therapeutic transmitted to the user device 100 may have the form of an application, and the user may download the digital therapeutic to the user device 100 and activate the digital therapeutic to perform a digital therapeutic-based treatment procedure. Data generated through the digital therapeutic-based treatment procedure may be transmitted back to the digital therapeutic generation device 120 as feedback data so that operations of the digital therapeutic may be controlled according to a change in the user

The digital therapeutic generation device 120 may generate a digital therapeutic. The digital therapeutic generation device 120 may generate a customized digital therapeutic on the basis of multi-source data analysis and provide the customized digital therapeutic to the user. The digital therapeutic generation device 120 may generate the digital therapeutic by combining a plurality of different digital therapeutic modules on the basis of the user data.

Also, the digital therapeutic generation device 120 may regenerate the provided digital therapeutic on the basis of the feedback data received from the user and transmit the regenerated digital therapeutic to the user device 100.

The information security device 140 may be implemented for the security of information generated by the user device 100 and the digital therapeutic generation device 120.

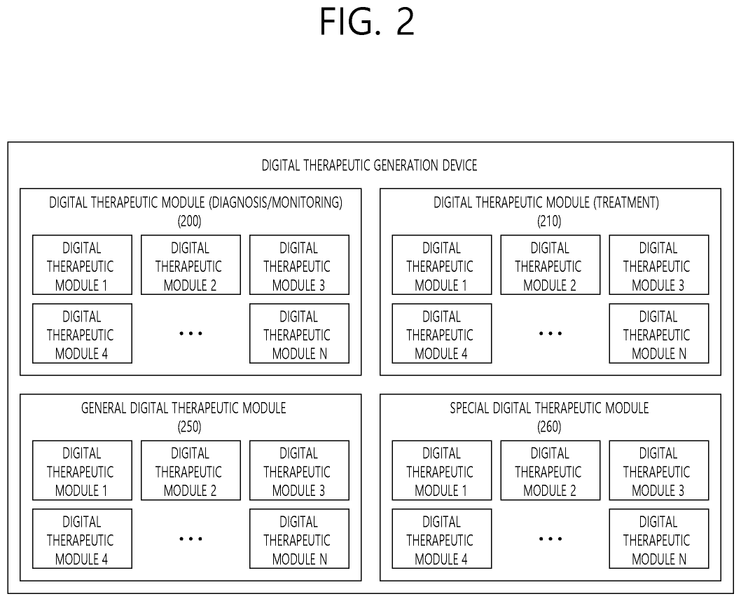

FIG. 2 is a conceptual diagram illustrating operations of a digital therapeutic generation device according to an exemplary embodiment of the present invention.

FIG. 2 shows operations for generating a digital therapeutic by combining a plurality of different digital therapeutic modules.

Referring to FIG. 2, digital therapeutic modules may be defined in individual function units that may be used for diagnosing, monitoring, and treating a user's disease.

Digital therapeutic modules may be classified as a digital therapeutic module (diagnosis/monitoring) 200 for diagnosis and monitoring, a digital therapeutic module (treatment) 210 for treatment, and the like.

For example, the digital therapeutic module (diagnosis/monitoring) 200 may include an activity check module for checking activity of the user, a sleeping hour check module for checking the user's sleeping hours, a diet check module for checking the user's diet, a temperature check module for checking the user's temperature, a biomarker check module for checking the user's biomarker, and the like.

The digital therapeutic module (treatment) 210 may include an optimal sleeping hour recommendation module for determining sleeping hours, a diet recommendation module for providing a recommendation for the user's diet, an administration recommendation module for providing information on the user's medicine to be taken, a dose of the medicine, and a timing of taking the medicine, and the like.

Also, according to an exemplary embodiment of the present invention, digital therapeutic modules may be classified as a general digital therapeutic module 250 and a special digital therapeutic module 260.

The general digital therapeutic module 250 may be a module that may be applied to several diseases in common to generate a digital therapeutic. The special digital therapeutic module 260 may be a module that may be used only for a specific disease to generate a digital therapeutic. For example, a digital therapeutic module for checking sleep, diet, and the like may be the general digital therapeutic module 250. A digital therapeutic module for managing a special disease (e.g., diabetes) may be the special digital therapeutic module 260.

The digital therapeutic generation device may combine the general digital therapeutic module 250 and the special digital therapeutic module 260 to generate a digital therapeutic.

The above digital therapeutic modules are examples. More diverse data therapeutic modules may be generated according to data, and an existing digital therapeutic may be adaptively changed with the addition of a digital therapeutic module and an improvement in performance.

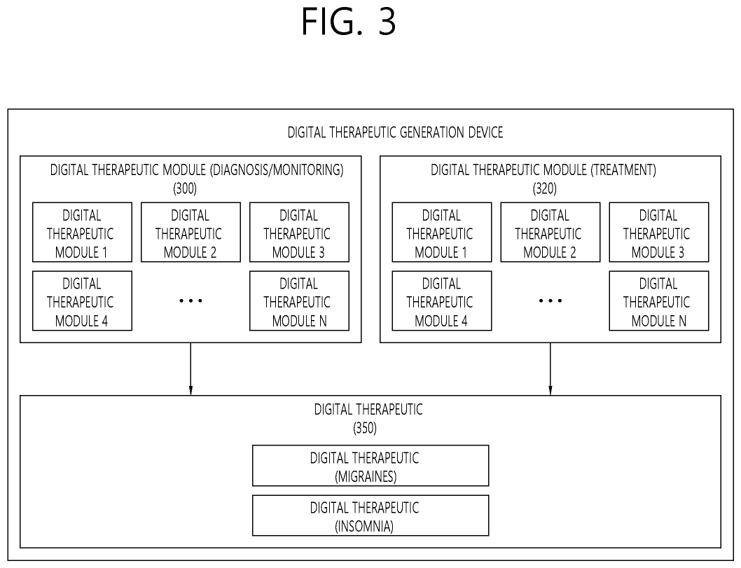

FIG. 3 is a conceptual diagram illustrating a method of generating a digital therapeutic according to an exemplary embodiment of the present invention.

FIG. 3 shows a method of generating a digital therapeutic by a digital therapeutic generation device.

Referring to FIG. 3, a digital therapeutic generation device may determine a digital therapeutic module (diagnosis/monitoring) 300 for diagnosing and monitoring a disease and a digital therapeutic module (treatment) 320 for treating a disease.

For example, in the case of insomnia, a digital therapeutic module (diagnosis/monitoring) 300 for monitoring a user's sleep state, activity state, and the like and a digital therapeutic module (treatment) 320 for managing the user's life pattern may be combined to generate a digital therapeutic 350 for insomnia.

As another example, in the case of migraines, a digital therapeutic module (diagnosis/monitoring) 300 for monitoring a user's sleep state, activity state, migraine period, surroundings, and the like and a digital therapeutic module (treatment) 320 for determining a medication, the amount of the medication to be taken, the timing of taking the medication, and the like may be combined to generate a digital therapeutic 350 for migraines.

The generated digital therapeutic may be provided to a user device, and a user's disease may be diagnosed/monitored and treated on the basis of the digital therapeutic provided to the user device.

Feedback data generated while the user uses the digital therapeutic may be transmitted to the digital therapeutic generation device, and the digital therapeutic may be adaptively changed on the basis of the feedback data and transmitted.

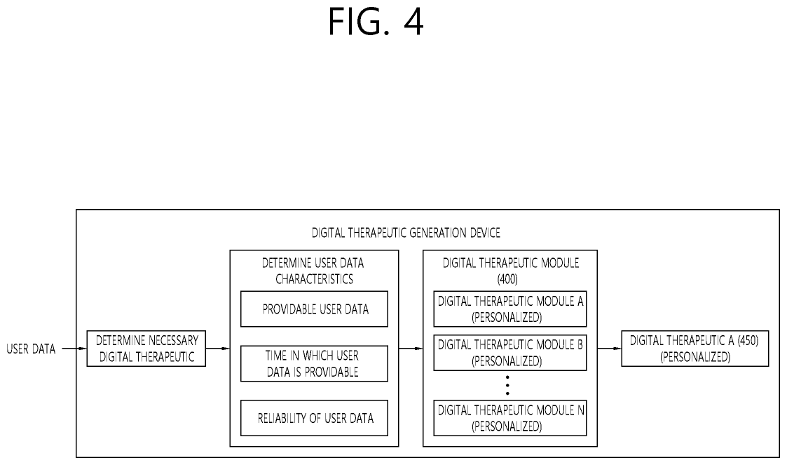

FIG. 4 is a conceptual diagram illustrating a method of generating a personalized digital therapeutic according to an exemplary embodiment of the present invention.

FIG. 4 shows a method of generating a personalized digital therapeutic by combining digital therapeutic modules.

Referring to FIG. 4, a digital therapeutic generation device may generate a personalized digital therapeutic by determining a personalized digital therapeutic module on the basis of an artificial intelligence engine.

The digital therapeutic generation device may determine a digital therapeutic required for a user on the basis of user data, combine digital therapeutic modules 400 suited for the digital therapeutic, and provide a digital therapeutic 450 personalized for the user.

For example, the digital therapeutic generation device may determine the user's sleep state and dietary habits on the basis of the user data and provide a digital therapeutic 450 obtained by combining digital therapeutic modules 400 for improving the user's sleep state and dietary habits. For a plurality of purposes (e.g., improving the sleep state and dietary habits), one digital therapeutic 450 personalized for the user may be provided, or a plurality of digital therapeutics 450 personalized for the user may be separately provided for the plurality of purposes.

The digital therapeutic generation device may generate a digital therapeutic in consideration of characteristics of user data that are providable by the user. For example, available digital therapeutic modules 400 may be selected from among digital therapeutic modules 400 and provided in consideration of whether specific data (heart rate data and blood pressure data) is providable, a time at which user data is providable, the reliability of the user data, and the like among the user data providable by the user.

As an example, when breathing data during sleep may be received as user data for the treatment of insomnia, the digital therapeutic generation device may include a digital therapeutic module x in a digital therapeutic 450, but when breathing data during sleep may not be received, the therapeutic generation device may include a digital therapeutic module y in a digital therapeutic 450. As another example, when a time period in which user data may be acquired is a first time period, the digital therapeutic generation device may include a digital therapeutic module z for providing digital treatment on the basis of only data about the first time period in a digital therapeutic 450 or may tune an existing digital therapeutic module 400 on the basis of a time at which the user's user data may be provided and include the tuned digital therapeutic module 400 in a digital therapeutic 450.

Also, it is necessary to make a judgment on a collision between a plurality of digital therapeutics (or digital therapeutic modules) in order to provide the personalized digital therapeutic 450 to the user.

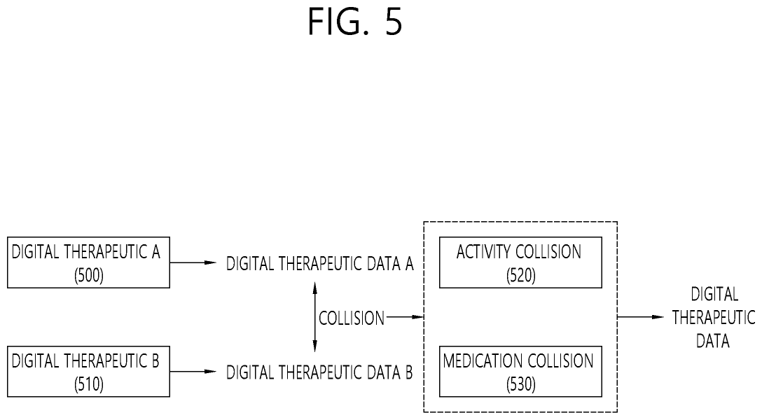

FIG. 5 is a conceptual diagram illustrating a digital therapeutic generation method according to an exemplary embodiment of the present invention.

FIG. 5 shows a method of processing a plurality of digital therapeutics when a collision occurs between the digital therapeutics.

Referring to FIG. 5, when a collision occurs between digital therapeutic data A generated by digital therapeutic A (500) and digital therapeutic data B generated by digital therapeutic B (510), digital therapeutic data of a digital therapeutic may be generated in consideration of the collision between the digital therapeutics.

Digital therapeutic A (500) may provide a diet algorithm for an eating disorder as digital therapeutic data on the basis of a digital therapeutic module, and digital therapeutic B (510) may provide a sleep algorithm for treating insomnia as digital therapeutic data on the basis of a digital therapeutic module. As an example, when a recommended sleep time of digital therapeutic B (510) overlaps a meal time of digital therapeutic A (500), because it is not possible to sleep and eat at the same time, the solutions generated by the digital therapeutics may collide with each other.

As another example, medication A to be taken which is proposed by a digital therapeutic for disease A may collide with medication B to be taken which is proposed by a digital therapeutic for disease B.

Therefore, when a plurality of digital therapeutics are provided to a user, the probability of a collision between digital therapeutic data of a plurality of digital therapeutics may be determined by a digital therapeutic generation device.

Collisions between digital therapeutic data of digital therapeutics may be classified as an activity collision 520, a medication collision 530, and the like according to the types of collision.

The activity collision 520 may correspond to a case where there is a collision between activities to be performed on the basis of digital therapeutic data of a plurality of digital therapeutics provided at a specific time. The digital therapeutic generation device may determine the probability of an activity collision at the same time through activity analysis between a plurality of pieces of digital therapeutic data provided to the user hourly.

The medication collision 530 may correspond to a case where there is a collision between medications to be taken on the basis of digital therapeutic data of a plurality of digital therapeutics provided at a specific time. The digital therapeutic generation device may determine the probability of a medication collision in consideration of a time to take a medication and a duration of the effects of the medication which are included in a plurality of pieces of digital therapeutic data provided to the user hourly.

The digital therapeutic generation device may determine the probability of the activity collision 520 and the medication collision 530 and adjust the digital therapeutic data when there is a probability of the activity collision 520 or the medication collision 530.

When there is a probability of the activity collision 520, the digital therapeutic generation device may determine an activity to be performed first on the basis of activity-specific priority positions. For example, the order of priority may be determined between a sleep time for insomnia and a meal time for an eating disorder. The order of priority may be determined in consideration of the severeness of the user's diseases and curative effects on the user caused by a corresponding activity. In other words, the order of priority may be determined differently for each user, and different digital therapeutic data may be provided to each user when the activity collision 520 occurs.

An activity with a higher priority position in the determined order of priority may be performed first when there is a collision between activities. Subsequently, an activity which has not been performed may not be performed in consideration of characteristics of the activity, or a time to perform the activity may be adjusted so that the activity may be performed at another time.

When there is a probability of the medication collision 530, the digital therapeutic generation device may determine whether it is possible to avoid the collision through medication-specific dosage adjustment. When it is possible to avoid the collision through dosage adjustment, digital therapeutic data in which dosages are adjusted may be provided through the digital therapeutics. When it is not possible to avoid the collision through medication-specific dosage adjustment, a medication to be taken first may be determined in consideration of medication-specific priority positions. For example, the order of priority of medications may be determined in consideration of the severeness of the diseases and curative effects of corresponding medications.

In the case of a medication that is pushed to a lower priority in order to avoid such a collision between medications, a time to take the medication may be delayed.

According to an exemplary embodiment of the present invention, medication effects and medication side effects caused by a collision between a plurality of medications may be determined as follows.

To consider medication effects and medication side effects of a combination of a plurality of medications, a combined embedded value for a medication combination may be determined on the basis of individual embedded values of the plurality of medications.

An individual embedded value of a medication may be positioned in a two-dimensional (2D) embedded plane as medication constituent 1 {effects, side effects}, medication constituent 2 {effects, side effects}, . . . , and medication constituent n {effects, side effects}, on the basis of effects and side effects of medication constituents constituting the medication.

According to an exemplary embodiment of the present invention, the combined embedded value for predicting a collision in consideration of effects and side effects of a combined medication may be generated on the basis of a combination of individual embedded values. Here, the combined embedded value may be determined in consideration of individual embedded values, concentration of the individual embedded values, and relationships between the individual embedded values.

First operation: Individual embedded values of a plurality of medications may be positioned in an embedded plane to determine a combined embedded value. When a specific one of the individual embedded values has a non-strong characteristic, that is, a threshold effect or less or a threshold side effect or less, the specific individual embedded value may be excluded.

Second operation: The individual embedded values of the plurality of medications may be adjusted in consideration of whether effects are changed (e.g., reduced effects or enhanced effects) due to a combination of medications on the basis of the relationships between individual embedded values or whether side effects are changed (e.g., reduced side effects or enhanced side effects) due to a combination of medications. For example, individual embedded values representing the degree of effects and the degree of side effects may be adjusted in consideration of the case where effects are enhanced, the case where side effects are reduced, and the like so that the positions in the embedded plane may be adjusted.

Third operation: In the combined embedded value, clusters reflecting effects and side effects may be made by clustering the individual embedded values. A dense cluster having a threshold concentration or more may be determined on the basis of concentrations of individual embedded values in the clusters. Clustering may be performed for each effect or each side effect. In other words, a clustering range is set to cluster the same or similar effects or the same or similar side effects so that a dense cluster may be determined among effects and among side effects

A combined embedded value may be finally determined on the basis of a concentration of individual embedded values in the dense cluster, and a prediction may be made on enhancement or reduction of a specific side effect and enhancement or reduction of a specific effect when a combination of a plurality of medications is taken. For example, when the concentration of a dense cluster corresponding to a specific effect is a threshold concentration or more, it is possible to predict enhancement of the effect, and when the concentration of a dense cluster corresponding to a specific side effect is the threshold concentration or more, it is possible to predict enhancement of the side effect.

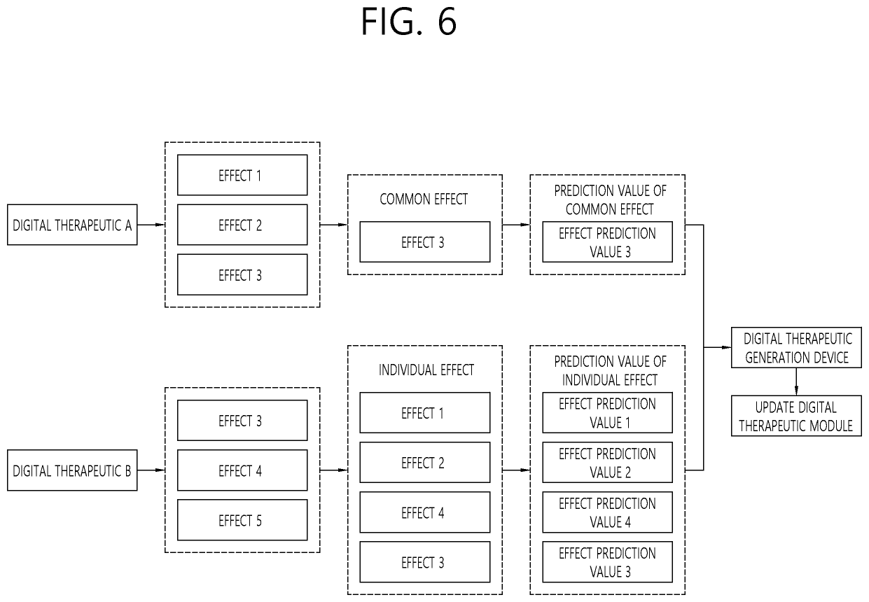

FIG. 6 is a conceptual diagram illustrating a method of managing a digital therapeutic for a user according to an exemplary embodiment of the present invention.

FIG. 6 shows a method of managing a digital therapeutic for a user.

Referring to FIG. 6, a plurality of digital therapeutics may be managed in a user device, and changes in disease symptoms made by a digital therapeutic and effects of the digital therapeutic may be separately managed.

For example, the user may manage disease A and disease B on the basis of digital therapeutic A and digital therapeutic B. It may be assumed that effect 1, effect 2, and effect 3 are expected on the basis of digital therapeutic A and effect 3, effect 4, and effect 5 are expected on the basis of digital therapeutic B. In this case, effect 3 may be set as a common effect, and effect 1, effect 2, effect 4, and effect 5 may be set as individual effects.

The individual effects can be fed back to a digital therapeutic generation device as effects based on digital therapeutic A or digital therapeutic B. The common effect can be fed back to the digital therapeutic generation device as an effect based on both digital therapeutic A and digital therapeutic B.

A prediction value can be set for each of such individual effects and common effects. The prediction values can be visually provided, and information about whether the prediction values are achieved using digital therapeutics can be visually provided. Accordingly, a user can visually check effects of the digital therapeutics.

After receiving information on effects/changes caused by treatment data as feedback, the digital therapeutic generation device can update a digital therapeutic by changing a digital therapeutic module involved in the digital therapeutic and provide the digital therapeutic to a user or can change treatment data provided on the basis of the digital therapeutic and provide the changed treatment data to the user.

The embodiments of the present invention described above may be implemented in the form of program instructions that can be executed through various computer units and recorded on computer readable media. The computer readable media may include program instructions, data files, data structures, or combinations thereof. The program instructions recorded on the computer readable media may be specially designed and prepared for the embodiments of the present invention or may be available instructions well known to those skilled in the field of computer software. Examples of the computer readable media include magnetic media such as a hard disk, a floppy disk, and a magnetic tape, optical media such as a compact disc read only memory (CD-ROM) and a digital video disc (DVD), magneto-optical media such as a floptical disk, and a hardware device, such as a ROM, a RAM, or a flash memory, that is specially made to store and execute the program instructions. Examples of the program instruction include machine code generated by a compiler and high-level language code that can be executed in a computer using an interpreter and the like. The hardware device may be configured as at least one software module in order to perform operations of embodiments of the present invention and vice versa.

While the present invention has been described with reference to specific details such as detailed components, specific embodiments and drawings, these are only examples to facilitate overall understanding of the present invention and the present invention is not limited thereto. It will be understood by those skilled in the art that various modifications and alterations may be made.

Therefore, the spirit and scope of the present invention are defined not by the detailed description of the present invention but by the appended claims, and encompass all modifications and equivalents that fall within the scope of the appended claims.

What is claimed is:

1. A method of generating a customized digital therapeutic on the basis of multi-source data analysis, the method comprising:
receiving, by a digital therapeutic generation device, user data; and
generating, by the digital therapeutic generation device, a digital therapeutic personalized for a user on the basis of the user data.

2. The method of claim 1, wherein the digital therapeutic generation device is generated on the basis of a combination of one or more digital therapeutic modules,
wherein the digital therapeutic modules include a digital therapeutic module (diagnosis/monitoring) and a digital therapeutic module (treatment).

3. The method of claim 2, wherein the digital therapeutic generation device is generated in consideration of a probability of a collision between digital therapeutics or characteristics of user data providable by the user.

4. A digital therapeutic generation device for generating a customized digital therapeutic on the basis of multi-source data analysis, the digital therapeutic generation device performing the operations of:
receiving user data; and
generating a digital therapeutic personalized for a user on the basis of the user data.

5. The digital therapeutic generation device of claim 4, wherein the digital therapeutic generation device is generated on the basis of a combination of one or more digital therapeutic modules,
wherein the digital therapeutic modules include a digital therapeutic module (diagnosis/monitoring) and a digital therapeutic module (treatment).

6. The digital therapeutic generation device of claim 5, wherein the digital therapeutic generation device is generated in consideration of a probability of a collision between digital therapeutics or characteristics of user data providable by the user.
